(19) United States
(12) Patent Application Publication
(10) Pub. No.: US 2015/0105650 A1
(43) Pub. Date: Apr. 16, 2015

(54) SIDE LOADING TORQUE DEVICES FOR INTRAVASCULAR DEVICES AND ASSOCIATED APPARATUS, SYSTEMS, AND METHODS

(71) Applicant: Volcano Corporation, San Diego, CA (US)

(72) Inventor: David H. Burkett, Temecula, CA (US)

(21) Appl. No.: 14/516,187

(22) Filed: Oct. 16, 2014

Related U.S. Application Data
(60) Provisional application No. 61/891,640, filed on Oct. 16, 2013.

(51) Int. Cl. A61M 25/01; A61B 1/313; A61M 25/09

(52) U.S. Cl. CPC A61M 25/0113; A61M 25/09041; A61B 1/3137; A61B 2017/00469
USPC 600/407; 604/95.01; 604/510; 74/543

(57) ABSTRACT

Torque devices, systems, and methods are disclosed. In some embodiments, a torque device includes a body having a proximal portion, a distal portion, and a longitudinal axis. The body includes a slot extending along a length of the body parallel to the longitudinal axis. The slot extends from an exterior surface of the body to an interior surface of the body. The slot is sized and shaped to receive a flexible elongate member. The body includes an opening extending through the body perpendicular to the longitudinal axis and in communication with the slot. The torque device includes a closing mechanism movably coupled to the body. The closing mechanism is movable within the opening of the body between an open position that allows the flexible elongate member to be inserted into the slot and a locked position that fixedly secures the flexible elongate member to the torque device.

### CROSS-REFERENCE TO RELATED APPLICATION

The present application claims the benefit of the filing date of U.S. Provisional Application No. 61/891,640 filed Oct. 16, 2013. The entire disclosure of this provisional application is incorporated herein by this reference.

### TECHNICAL FIELD

The present disclosure relates to components used with intravascular devices. In some embodiments, the components are torque devices that allow efficient maneuvering of intravascular devices within a patient's vasculature by an operator.

### BACKGROUND

Torque devices are used in intravascular procedures to control the position of an intracellular device, such as a guide wire or catheter, within a vessel of a patient. A guide wire generally has a small, circular cross-section that can be difficult for an operator to grasp. Even when the guide wire can be grasped, it is difficult for an operator to apply torque and cause the guide wire to rotate (e.g., about a longitudinal axis of the guide wire) in a controlled manner. A torque device generally has a larger diameter and is easier for an operator to grasp and to apply torque to. The torque device may be coupled to a guide wire such that guide wire can be steered, maneuvered, and/or otherwise controlled inside the patient's vasculature by an operator. For example, the torque device may enable an operator to traverse a patient's vein or artery with the guide wire as the artery and/or the vein twists, turns, and/or otherwise deviates from a straight path (as is common within a patient's body). When the torque device is locked to the guide wire, the rotation of the guide wire and torque device are fixed such that rotation of the torque device causes rotation of the guide wire. Thus, using a torque device, an operator may rotate the guide wire to turn within an artery or vein, as the artery or vein turns within the patient's body.

Conventional torque devices are through-hole loading. This means that the torque device is loaded (and unloaded) over the proximal end of the guide wire and then slid along the length of the guide wire to a desired position. Conventional torque devices utilize a brass collet in an assembly that as the nose is screwed tight on the body, the brass collet's edges dig into the guide wire to create a grip on the guide wire. One shortcoming of the conventional design is that the edges of the brass collet can cut into coated outer surfaces of the guide wire (e.g., hydrophobic coatings, hydrophillic coatings, polytetrafluoroethylene (“PTFE”) coatings, etc.). This creates the potential for coating particulate to infiltrate the patient's body, which can lead to further medical complications for the patient.

Recent concepts for, e.g., pressure and flow guide wires (such as those described in U.S. Provisional Patent Application No. 61/665,711, filed Jun. 28, 2012 and U.S. Patent Application No. 61/665,739, filed Jun. 28, 2012, each of which is hereby incorporated by reference in its entirety) include embedded conductive ribbons and printed gold conductive bands. These new concepts contemplate that the traditional stainless steel hypotube of, e.g., the pressure or flow guide wire is eliminated, and that the external surface of the guide wire be a lubricious coated polymer material. In these types of guide wires, the use of brass collet torque devices is not acceptable because of the potential for damage to the conductive members when the torque device is clamped onto the wire. Some conventional torque devices utilize plastic collets that suffer from poor torqueability in use.

Conventional torque devices generally require holding the body of the torque device with one hand and rotating the nose of the torque device to loosen or tighten the collet onto the wire. As a result, every time the doctor needs to reposition the torque device, this two-handed sequence is required. This leads to inefficiencies during the intravascular procedure.

Accordingly, there remains a need for improved torque devices for use with intravascular devices (e.g., catheters and guide wires) that allow for efficient loading and unloading of the torque device, without damage to the intravascular devices, while providing good torqueability of the intravascular devices.

### SUMMARY

Embodiments of the present disclosure are directed to a torque device that allows for side-loading of the intravascular device (e.g., catheter and guide wire) and utilizes a wedge element that moves perpendicular to the intravascular device to lock the torque device onto the intravascular device.

In some embodiments, a torque device is provided. In one embodiment, the torque device includes a body having a proximal portion, a distal portion, and a longitudinal axis. The body includes a slot extending along a length of the body parallel to the longitudinal axis. The slot extends from an exterior surface of the body to an interior surface of the body. The slot is sized and shaped to receive a flexible elongate member. The body includes an opening extending through the body perpendicular to the longitudinal axis and in communication with the slot. The torque device includes a closing mechanism movably coupled to the body. The closing mechanism is movable within the opening of the body between an open position that allows the flexible elongate member to be inserted into the slot and a locked position that fixedly secures the flexible elongate member to the torque device.

In some embodiments, a system is provided. In one embodiment, the system includes an intravascular device sized and shaped for insertion within a vessel of a patient. The system includes a torque device configured to selectively, fixedly engage a proximal section of the intravascular device. The torque device includes a body having a proximal portion, a distal portion, and a longitudinal axis. The body includes a slot extending along a length of the body parallel to the longitudinal axis. The slot extends from an exterior surface of the body to an interior surface of the body. The slot is sized and shaped to receive at least the proximal section of the intravascular device. The body includes an opening extending through the body perpendicular to the longitudinal axis and in communication with the slot. The torque device includes a closing mechanism movably coupled to the body. The closing mechanism is movable within the opening of the body between an open position that allows the intravascular device to be inserted into the slot and a locked position that fixedly secures the intravascular device to the torque device.

In some embodiments, a method is provided. In one embodiment, the method includes inserting an intravascular device into a slot of a body portion of a torque device in a direction perpendicular to a longitudinal axis of the intravascular device. The method includes moving a closing mechanism of the torque device from an open position that allows the intravascular device to be inserted into the slot to a locked position that fixedly secures the intravascular device to the torque device. Moving the closing mechanism from the open position to the closed position includes translating the closing mechanism in a direction perpendicular to the longitudinal axis of the intravascular device along an opening in the body portion of the torque device that is in communication with the slot.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic perspective view of an intravascular system according to an embodiment of the present disclosure.

FIG. 2 is a diagrammatic perspective view of a torque device according to an embodiment of the present disclosure.

FIG. 3 is a diagrammatic cross-sectional perspective view of a torque device according to an embodiment of the present disclosure.

FIG. 4 is a diagrammatic back view of a torque device according to an embodiment of the present disclosure.

FIG. 5 is a diagrammatic front view of a torque device according to an embodiment of the present disclosure.

FIG. 6 is a diagrammatic top view of a wedge according to an embodiment of the present disclosure.

FIG. 7 is a diagrammatic back view of a wedge according to an embodiment of the present disclosure.

FIG. 8 is a diagrammatic top view of an intravascular system according to an embodiment of the present disclosure.

FIG. 9 is a diagrammatic cross-sectional back view of an intravascular system according to an embodiment of the present disclosure.

FIG. 10 is diagrammatic perspective views of intravascular systems according to embodiments of the present disclosure.

FIG. 11 is diagrammatic perspective views of intravascular systems according to embodiments of the present disclosure.

FIG. 12 is a chart describing torqueability of an intravascular system according to an embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, intravascular catheters and intravascular guide wires. In that regard, intravascular catheters may or may not include a lumen extending along its length for receiving and/or guiding other instruments. If the intravascular catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a temperature sensor, an imaging element, an optical fiber, an ultrasound transducer, a reflector, a mirror, a prism, an ablation element, an electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of the intravascular space, particularly the interior walls of human vasculature can be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized. Further, in some instances the flexible elongate member includes multiple electronic, optical, and/or electro-optical components (e.g., pressure sensors, temperature sensors, imaging elements, optical fibers, ultrasound transducers, reflectors, mirrors, prisms, ablation elements, fro electrodes, conductors, etc.).

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members can be solid, some embodiments of the present disclosure will include a housing portion at the distal portion for receiving the electronic components. Such housing portions can be tubular structures attached to the distal portion of the elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the elongate member, such as a guide wire or catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm) and approximately 0.035″ (0.889 mm). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

According to one or more aspects of the present disclosure, a side-loading torque device is provided. A linear wedge is incorporated into the side-loading design. The wedge includes a flat side and an at least partially tapered/sloped side. The wedge moves perpendicular to the axis of the flexible elongate member. The tapered side contacts the flexible elongate member and wedges it into a grooved receptacle (e.g., a slot) in the torque device. Locking the torque device onto the flexible elongate member is accomplished by pushing the wedge, which is at least partially received within the torque device, in one direction. Unlocking is accomplished by pushing or advancing the wedge in an opposing direction. Positioning the wedge between the locked and unlocked positions can be utilized to keep the flexible elongate member retained in the torque device but allow for easy repositioning by sliding the torque device to a new location along the length of the flexible elongate member.

According to one or more aspects of the present disclosure, retention features are incorporated into the body of the torque device and/or the wedge that retain the wedge at least partially in or coupled to the torque device, even when no flexible elongate member is positioned within the torque device. Projections, detents, and/or combinations thereof on the wedge and/or the torque device may be utilized to allow the wedge to be in a partially open position without allowing the flexible elongate member to be removed from the torque device so that the torque device can be easily repositioned along the length of the flexible elongate member.

According to one or more aspects of the present disclosure, the side-loading design advantageously provides more efficient loading and/or unloading of the torque device at a desired position along the length of the flexible elongate member. The side-loading torque device provides an operator (e.g., a physician) the ability to quickly load and unload the torque device at the desired position by eliminating the need to slide the torque device over the flexible elongate member (e.g., as in conventional, through-loading torque devices). Direct side-access (e.g., direct drop-in) is advantageously provided with the side-loading design.

According to one or more aspects of the present disclosure, the wedge design advantageously provides a way of locking the torque device onto the flexible elongate member without damaging the surface of the flexible elongate member. The torque device and wedge may be formed of a plastic material. The wedge greatly reduces or eliminates the potential damage and particulate formation that arises with the use of conventional brass collet torque devices on polymer coated intravascular devices. The torque device described herein can be used with any flexible elongate member, regardless of whether or not the flexible elongate member includes electronic components. Flexible elongate members with or without electronic components can include coatings that are damaged less using the torque device described herein. The wedge design also allows for easier and more efficient loading/unloading of the torque device onto the intravascular device. One-handed operation to load, unload, lock, unlock, and/or move the torque device is also possible with the embodiments of the present disclosure. The wedge design also advantageously allows good torqueability because the wedge provides stronger locking onto the flexible elongate member than conventional plastic collet designs. For example, the wedge designs of the present disclosure provide longer and/or larger contact surfaces between the torque device and the flexible elongate member and eliminate sharp plastic edges that are easily deformed.

One or more embodiments of the torque devices of the present disclosure include only two molded components, which is advantageously more cost efficient to produce than conventional torque devices that include a machined brass collet in addition to two molded components.

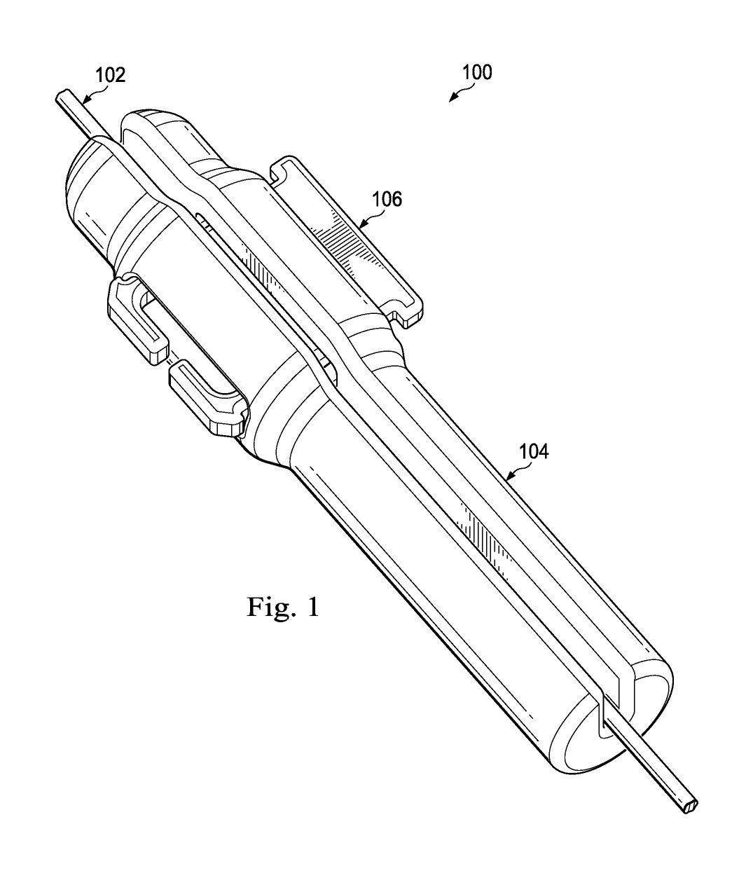

Referring now to FIG. 1, a diagrammatic perspective view of an intravascular system is shown, according to an embodiment of the present disclosure. Intravascular system 100 includes a flexible elongate member 102. In the embodiment shown of FIG. 1, a portion of the flexible elongate member 102 nearer to the proximal end of flexible elongate member 102 is shown. The more distal portions of flexible elongate member 102 may be inserted into the vasculature of a patient. Intravascular system 100 also includes a torque device 104. When coupled to and/or locked on flexible elongate member 102, torque device 104 facilitates traversal of a patient's veins or arteries by the flexible elongate member. Flexible elongate member 102 and torque device 104 may be coaxially disposed. Torque device 104 includes a wedge 106, which locks, grips, and/or otherwise secures flexible elongate member 102 and torque device 104 together.

In some embodiments, more distal portions of flexible elongate member include one or more electronic, optical, or electro-optical components. In that regard, the component is a pressure sensor, a temperature sensor, an imaging element, an optical fiber, an ultrasound transducer, a reflector, a mirror, a prism, an ablation element, an fro electrode, a conductor, and/or combinations thereof. The specific type of component or combination of components can be selected based on an intended use of the intravascular device. In some instances, the component is positioned less than 10 cm, less than 5, or less than 3 cm from the distal tip of the flexible elongate member. In some instances, the component is positioned within a housing of the flexible elongate member 102. In that regard, the housing is a separate component secured to the flexible elongate member 102 in some instances. In other instances, the housing is integrally formed as a part of the flexible elongate member 102. In some embodiments, the flexible elongate member 102 comprises a stainless steel hypotube or a polymer tubing. Further, in some embodiments all or a portion of the flexible elongate member 102 is covered with a hydrophilic or hydrophobic coating. In some particular embodiments, a polytetrafluoroethylene (“PTFE”) coating is utilized.

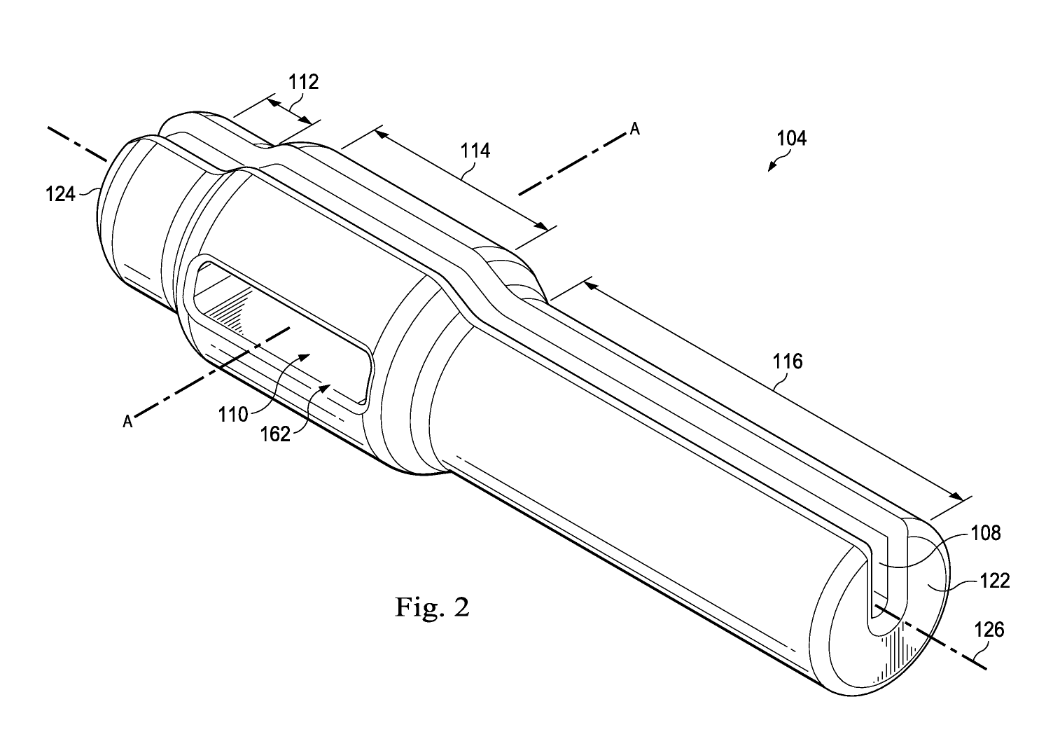

Referring now to FIG. 2, a diagrammatic perspective view of a torque device is shown, according to an embodiment of the present disclosure. As described herein, torque device 104 is configured to be engaged with, loaded onto, and/or coupled to a flexible elongate member. For example, the flexible elongate member may be received within slot 108 of torque device 104. A wedge or locking member may be received in bore 110 of torque device 104 while flexible elongate member is disposed in slot 108. Contact between the wedge or locking member and the flexible elongate member fixedly secures the torque device 104 and flexible elongate member together.

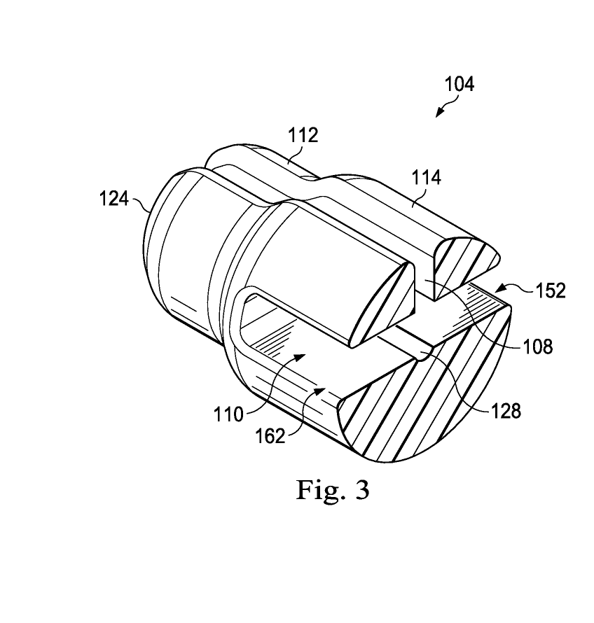

Torque device 104 is shown to have a generally cylindrical shape. That is, a cross-section of torque device 104 along a plane perpendicular to longitudinal axis 126 of torque device 104 is generally circular (as shown in, e.g., FIG. 3). In various embodiments, the general shape of torque device 104 may be different (e.g., polyhedron, spheroid, etc.). It is understood that the torque device 104 may have any shape suitable for handheld use, including symmetrical shapes, non-symmetrical shapes, geometric shapes, non-geometric shapes, and/or combinations thereof. Torque device 104 may be variously referred to as a first member and/or a body member in the discussion herein.

Torque device 104 includes a distal section 112, a central section 114, and a proximal section 116. In the embodiment of FIG. 2, the distal section 112 is shown to have length (e.g., an extent along longitudinal axis 126) less than central section 116, which has a length less than proximal portion 116. In some embodiments, the individual lengths of sections 112, 114, 116 may vary, and the relative lengths of sections 112, 114, 116 may also vary (e.g., distal section 112 may have the same or nearly same length as central portion 114, each section may have the same or nearly same length, etc.) In some embodiments, torque device 104 may not have distinguishable sections, such as when length and/or radius of sections 112, 114, 116 are equal or nearly equal.

In FIG. 2, each section 112, 114, 116 is shown to be similarly shaped (e.g., each section is itself generally cylindrical). In some embodiments, one or more of sections 112, 114, 116 may be differently shaped (e.g., distal section 112 may be spheroidal, central section 114 may be a rectangular prism, etc.). In some embodiments, the shapes of the sections 112, 114, 116 are selected to facilitate convenient hand-held grasping by a user.

In FIG. 2, the radius of central section 114 is shown to be larger than the radii of proximal section 116 and distal section 112. The radii of proximal section 116 and distal section 112 are shown to be equivalent or nearly equivalent. In some embodiments, the individual radii of sections 112, 114, 116 may vary, and the relative radii of sections 112, 114, 116 may also vary (e.g., the radius of proximal section 116 may be greater than the radius of distal section 112, the radius of proximal section 116 may be equivalent or nearly equivalent to the radius of central section 114, etc.) For example, in some embodiments, the radii of sections 112, 114, 116 may all be equal or nearly equal.

Torque device 104 is shown to include transition zones as the radius changes between sections 112, 114 and between sections 114, 116. The gradual change of the outer diameter of the torque device is reflected in the transition zones. In some embodiments, the transition zones may be larger (e.g., a more gradual changes in outer diameter) or shorter (e.g., less gradual change in other diameter). In some embodiments, torque device 104 may include no transition zones (e.g., the outer diameter changes are immediate, stepped changes; the radius of the torque device is uniform throughout; etc.).

The proximal section 116 includes a rounded and/or tapered portion as it transitions to proximal end 122 (also shown in, e.g., FIGS. 1, 4, 10, 11). Similarly, the distal section 112 includes a rounded and/or tapered portion as it transitions to distal end 124 (also shown in, e.g., FIGS. 1, 5, 10, 11). In some embodiments, proximal end 122 and/or distal end 124 may shaped differently. Likewise, the transition between the proximal section 116 and the proximal end 122 and/or the transition between the distal section 112 and the distal end 124 the proximal end 122 may have a different profile (e.g., more or less tapered, more or less arcuate, and/or other changing profile).

In one aspect of the present disclosure, the length(s) and diameter(s) of the torque device 104 are selected to allow for single-handed use. For example, a short, smaller diameter distal section 112 (e.g., a nose section) can provide a finger support when locking or unlocking the wedge, while the outer diameter of the proximal section 116 can be sized such that it is familiar to an operator (e.g., similar in size to existing torque devices). In some embodiments, proximal section 116 is the area that the operator will hold onto when steering the flexible elongate member 102.

One or more of sections 112, 114, 116 may include additional features to facilitate an operator's grasp of torque device 104. For example, one or more longitudinal ribs may be provided on at least a portion of sections 112, 114, and/or 116. For example, one or more flat and/or recessed portions (e.g., a cut out of one or more of sections 112, 114, 116) may be provided for an operator's fingers (e.g., thumb and pointer finger) to rest while grasping the torque device 104.

In the embodiment of FIG. 2, the exterior surface of torque device 104 is smooth. In other embodiments, one or more texture elements may be provided to facilitate an operator's grasp of torque device 104. For example, one or more longitudinal ribs spanning at least a portion of the exterior surface of torque device 104 are provided. For example, raised notches, roughened texture, knurled texture, and/or other patterns may be provided on all or some portions of proximal section 116.

In some embodiments, torque device 104 may be integrally formed. In other embodiments, torque device 104 may be a modular assembly including one or more pieces. For example, one or more of sections 112, 114, 116 may be individual components that are coupled together to form torque device 104.

Torque device 104 includes a slot 108. Slot 108 may be variously referred to a first slot, longitudinal slot, channel, and/or groove in the description herein. Slot 108 extends longitudinally along an entire length of the torque device 104 (e.g., along and/or parallel to the longitudinal axis 126). Thus, slot 108 may extend through sections 112, 114, 116 of torque device 104. Slot 108 extends from an exterior surface of torque device 104 to an interior surface of torque device 104. Slot 108 may be sized and shaped to receive a flexible elongate member (e.g., flexible elongate member 102 of FIG. 1). Slot 108 extends along the length of the torque device 104 such that the torque device 104 may be side loaded onto the flexible elongate member. That is, torque device 104 may be engaged with a flexible elongate member in a direction perpendicular to the longitudinal axes of both the torque device and the flexible elongate member such that a portion of the flexible elongate member is seated within slot 108. An operator is advantageously able to select a location along flexible elongate member 102 at which to load torque device 104 and then side load the torque device at that location. This eliminates the need to slide the torque device 104 from the proximal end of the flexible elongate member along the length of the flexible elongate member to the desired location. However, the torque device 104 can be loaded onto the flexible elongate member in this traditional manner, if desired. The surfaces defining slot 108 may be smooth such that wear and/or other damage does not occur to either of the flexible elongate member or slot 108 during use. In other embodiments, the surfaces defining slot 108 may be textured such that flexible elongate member is at least partially maintained within slot 108 via contact between the texture elements and the exterior surface of the flexible elongate member.

Torque device 104 includes a bore 110. Bore 110 may be variously referred to as a second slot or a locking channel in the description herein. Bore 110 may be referred to as part of a fastening or closing mechanism of the torque device 104. Bore 110 extends across an entire width of torque device 104 in a direction perpendicular to the longitudinal axis 126 and slot 108 (e.g., parallel to section line A-A). Thus, bore 110 may extend through torque device 104 (e.g., transverse slot 108), including through a space in torque device 104 defined by slot 108. Left opening 162 defines one side of the bore 110. (Right opening 152 of, e.g., FIG. 3 defines another side of bore 110.) According to an exemplary embodiment, bore 110 is contained entirely in central section 114. In other embodiments, bore 110 may extend into one or both of distal section 112 and proximal section 116. Bore 110 may be sized and shaped to receive a wedge (e.g., wedge 106 of FIG. 1).

In use, torque device 104 may receive at least a portion of a flexible elongate member (e.g., flexible elongate member 102 of FIG. 1) in slot 108. The flexible elongate member may be coupled to torque device 104 via a fastening or closing mechanism, including a wedge (e.g., wedge 106 of FIG. 1). The wedge may be inserted in bore 110 such that contact between the wedge and flexible elongate member locks the torque device to the flexible elongate member.

Referring now to FIG. 3, a diagrammatic cross-sectional perspective view of a torque device is shown, according to one embodiment of the present disclosure. FIG. 3 is a cross-sectional view of torque device 104 along section line A-A of FIG. 2. As described herein, torque device 104 is side loaded onto a flexible elongate member such that the flexible elongate member is received in slot 108. A wedge or locking member is inserted through bore 110, which extends across torque device 104 in communication with the slot 108. Contact between the wedge or locking member and the flexible elongate member locks torque device 104 to the flexible elongate member.

FIG. 3 includes distal section 112, which terminates at distal end 124. FIG. 3 also includes a portion of central section 114. Slot 108 is disposed longitudinally along the length of torque device 104. Slot 108 extends from an exterior surface of torque device 104 to an interior bottom surface 128 of slot 108. When a flexible elongate member (e.g., flexible elongate member 102 of FIG. 1) is coupled to torque device 104, the flexible elongate member may be seated in bottom surface 128 of slot 108. Bottom surface 128 may be variously described as the innermost extent of slot 108 into an interior of torque device 104 or as a groove within the interior of torque device 104. Bore 110 is shown to extend perpendicularly to slot 108 and a longitudinal axis of torque device 104. Slot 108 and bore 110 may intersect in an interior of torque device 104 such that there is a shared space that is part of both slot 108 and bore 110. Bore 110 extends across an entire width of torque device 104 between left opening 162 and right opening 152. A cross-section of bore 110 is shown to be generally rectangular or trapezoidal. In various embodiments, the shape and dimensions of bore 110 may vary, e.g., to accommodate a wedge (e.g., wedge 106 of FIG. 1), which itself may vary in shape and dimensions. The surfaces of bore 110 are shown to be smooth. In various embodiments, the surface of bore 110 may be textured such that there contact between exterior surfaces of the wedge and the texture elements on the surfaces of bore 110.

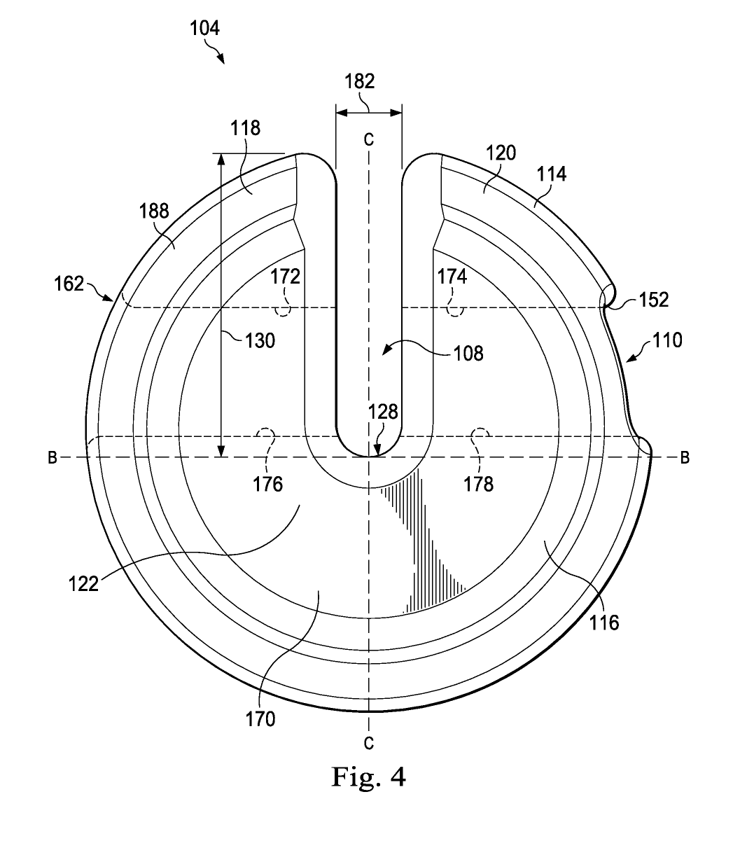

Referring now to FIG. 4, a diagrammatic back view of a torque device is shown, according to one embodiment of the present disclosure. FIG. 4 is a view of torque device 104 from proximal end 122. As shown, proximal section 116 and central section 114 of torque device 104 are visible in FIG. 4. According to an exemplary embodiment, a smoothly-changing outer diameter is provided in the transition zone between proximal section 116 and central section 114. The rounded transition between proximal section 116 and proximal end 122 is also shown in FIG. 4. In some embodiments, the transition between proximal section 116 and proximal end 122 may be tapered, arcuate, and/or other changing profile.

FIG. 4 includes section lines B-B and C-C. Section line B-B divides torque device 104 into top and bottom halves. Section line C-C devices torque device 104 into left and right halves. A lower portion 170 of torque device 104 (e.g., the parts of torque device 104 below section line B-B) is shown to be undivided. An upper portion 188 of torque device 104 (e.g., the parts of torque device 104 above section line B-B) is bifurcated by slot 108. Slot 108 is disposed between left portion 118 (e.g., the parts of torque device 104 to the left of section line C-C) and right portion 120 (e.g., the parts of torque device 104 to the right of section line C-C). Upper portion 188 thus includes left portion 118 and right portion 120. In some embodiments, slot 108 equally or nearly equally divides upper portion 188 of torque device 104 into the left portion 118 and the right portion 120 (e.g., such that left portion 118 and right portion 120 are mirror images of each other). In some embodiments, slot 108 may be offset to the right or left such that the width of one of left portion 118 and right portion 120 is greater than the width of the other. In some embodiments, slot 180 is disposed parallel to section line C-C. In some embodiments, slot 180 is disposed at an angle relative to section line C-C towards either the left portion 118 or the right portion 120. Slot 108 extends along an entire length of torque device 104 such that torque device 104 may be side loaded onto a flexible elongate member via a lateral side (e.g., the lateral side with slot 108) of torque device 104.

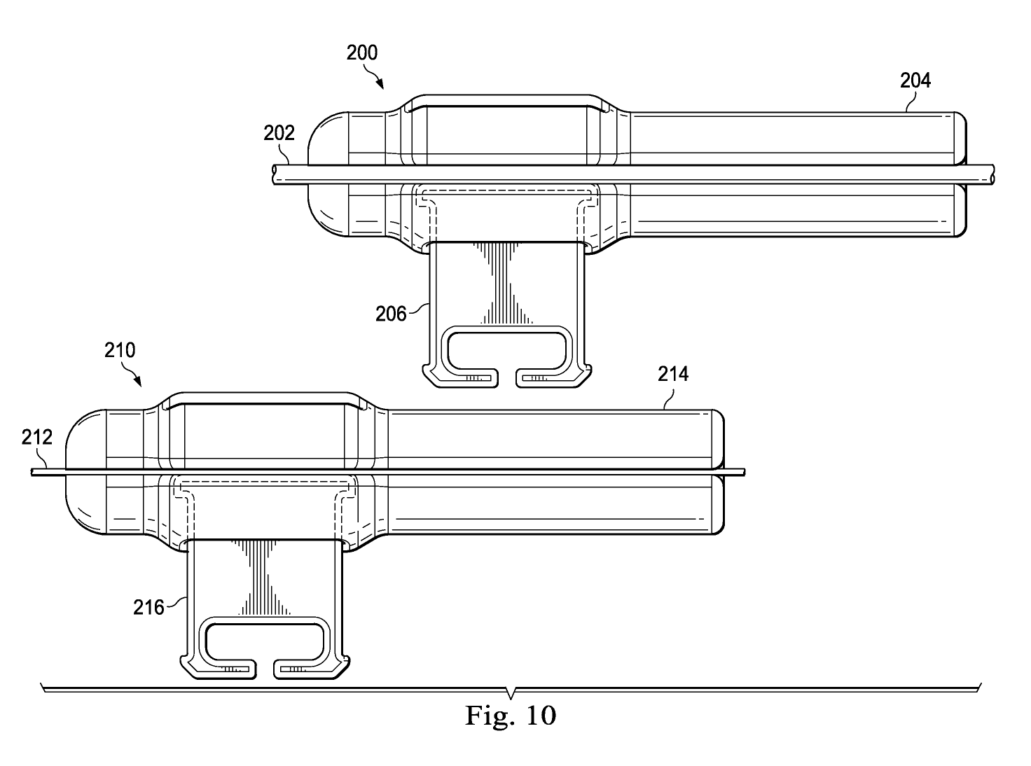

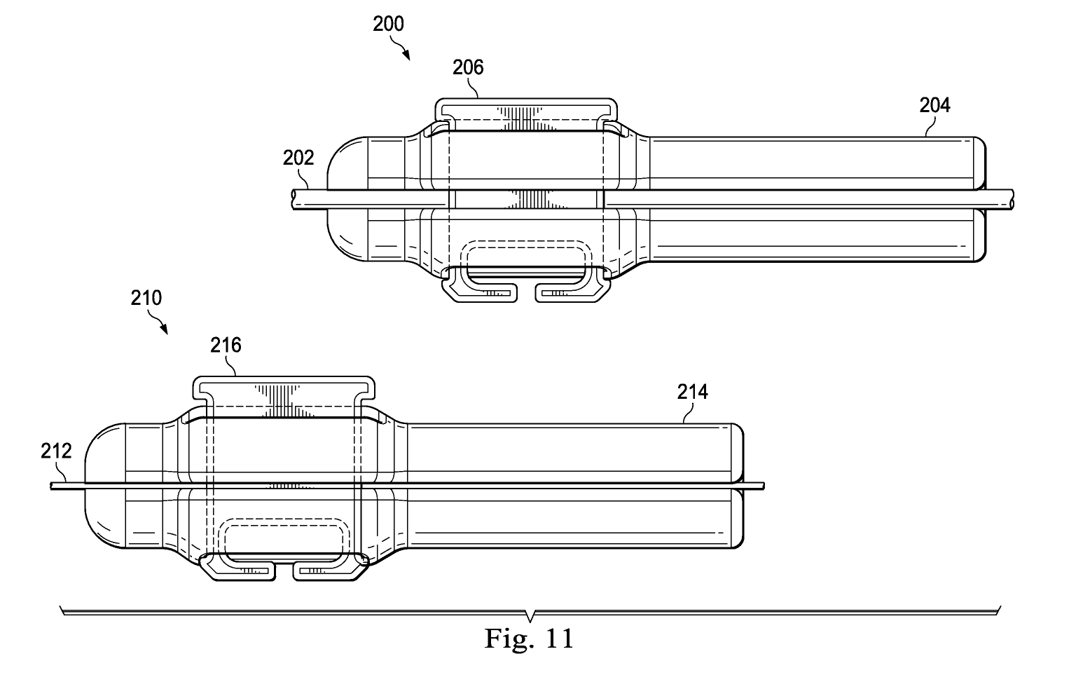

Slot 108 may have depth 130 describing an extent into an interior of torque device 104 that slot 108 extends. Slot 108 is shown to extend into the center or nearly the center of torque device 104 (as viewed in perspective of FIG. 4). Bottom surface 128 may represent the farthest distance into an interior of torque device 104 that slot 108 extends. Slot 108 may have a width 182 describing an extent of separation between left portion 118 and right portion 120. Depth 130 and/or width 182 may be variously chosen to accommodate flexible elongate members of different sizes (as shown in, e.g., FIGS. 10, 11).

Bore 110, which extends through torque device 104, is shown in phantom in FIG. 4. Bore 110 is disposed perpendicular to and/or transverse to slot 108 and the longitudinal axis of torque device 104. Surfaces 172, 174, 176, 178 of bore 110 are also shown. Surface 172 is a top surface of bore 110 in the left portion 118. Surface 174 is a top surface of bore 110 in the right portion 120. Surface 176 is a bottom surface of bore 110 in the left portion 118. Surface 178 is a bottom surface of bore 110 in the right portion 120. Right opening 152, which defines one side of bore 110, is disposed to the right of section line C-C in FIG. 4. Left opening 162, which defines the other side of bore 110, is disposed to the left of section line C-C. In some embodiments, right opening 152 is larger than left opening 162. In some embodiments, right opening 152 and left opening 162 are similarly sized.

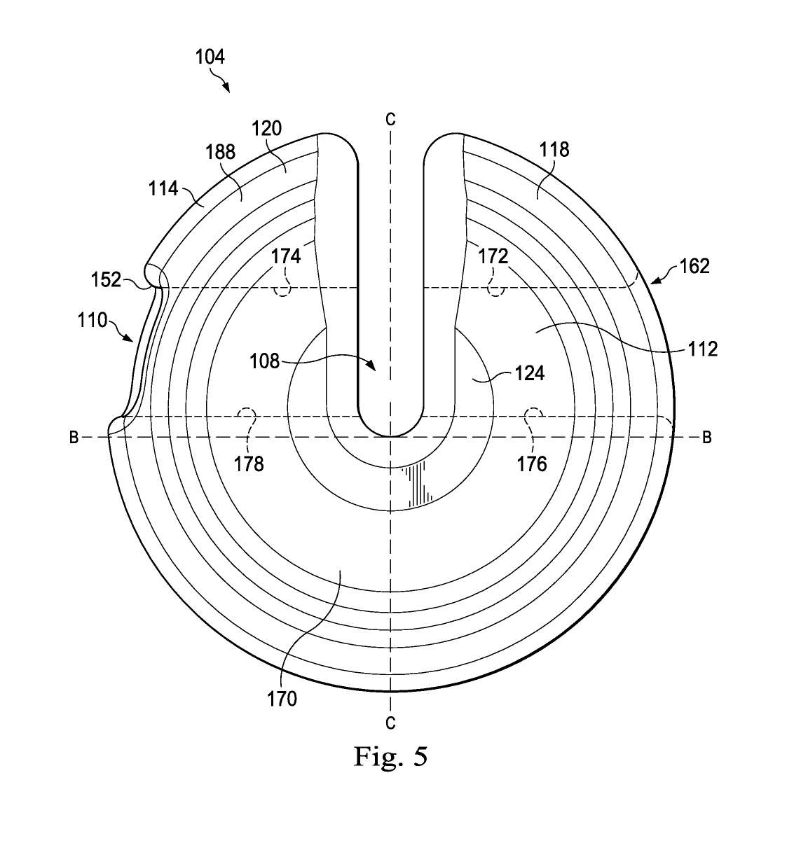

Referring now to FIG. 5, a diagrammatic front view of a torque device is shown, according to one embodiment of the present disclosure. FIG. 5 is a view of torque device 104 from distal end 124. FIG. 5 includes distal section 112 and central section 114. According to an exemplary embodiment, a smoothly-changing outer diameter is provided in the transition zone between distal section 112 and central section 114. Distal section 112 is shown to be tapered and/or rounded to distal end 124. In some embodiments, the transition between distal section 112 and distal end 124 may be tapered, arcuate, and/or other changing profile.

FIG. 5 includes section lines B-B and C-C. As in FIG. 4, section line B-B divides torque device 104 into top and bottom halves, and section line C-C devices torque device 104 into left and right halves. Lower portion 170 (e.g., parts of torque device 104 below section line B-B) is shown to be undivided. Upper portion 188 (e.g., parts of torque device 104 above section line B-B) is bifurcated by slot 108. Slot 108 is disposed between right portion 118 (e.g., parts of torque device 104 to the right of section line C-C) and left portion 120 (e.g., parts of torque device 104 to the left of section line C-C). Upper portion 188 thus includes left portion 118 and right portion 120. Bore 110, which extends through torque device 104, is shown in phantom in FIG. 5. Bore 110 is disposed perpendicular to and/or transverse to slot 108 and the longitudinal axis of torque device 104. Surfaces 172, 174, 176, 178 of bore 110 are also shown. Surface 172 is a top surface of bore 110 in the left portion 118. Surface 174 is a top surface of bore 110 in the right portion 120. Surface 176 is a bottom surface of bore 110 in the left portion 118. Surface 178 is a bottom surface of bore 110 in the right portion 120. Right opening 152, which defines one side of bore 110, is disposed to the left of section line C-C in FIG. 4. Left opening 162, which defines the other side of bore 110, is disposed to the right of section line C-C. One or more features described in the discussion of FIG. 4 is similarly shown in FIG. 5.

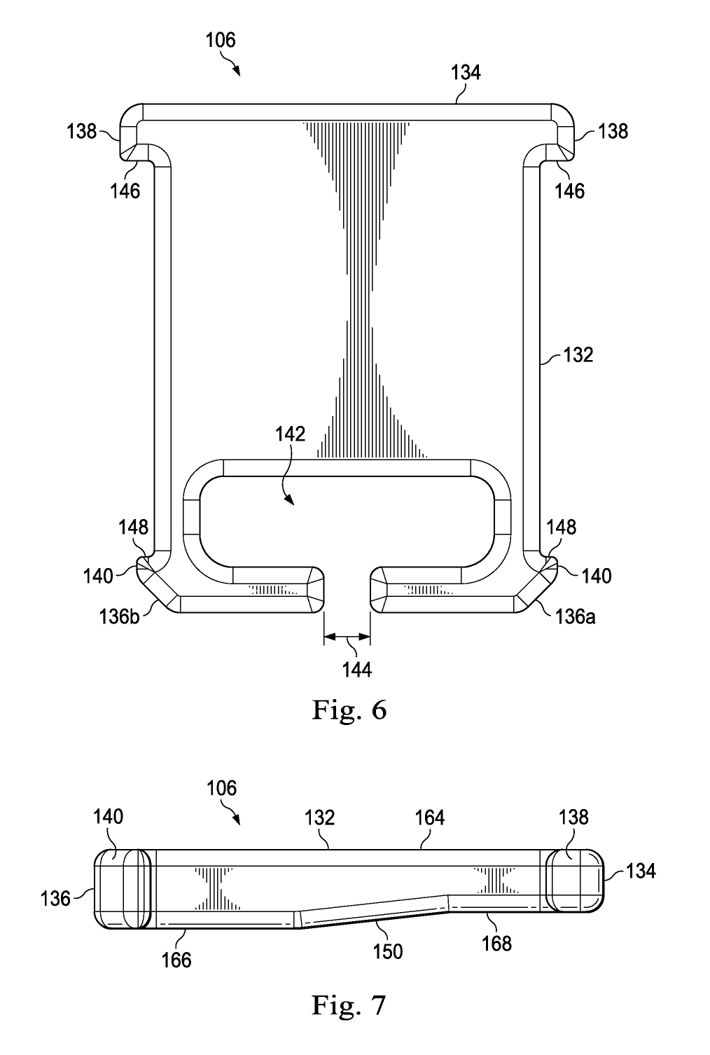

Referring now to FIG. 6, a diagrammatic top view of a wedge is shown, according to one embodiment of the present disclosure. As described in more detail herein, wedge 106 may be received in and translate within the bore (e.g., bore 110) of torque device 104. Wedge 106 may be a locking member and/or part of a locking mechanism. Contact between wedge 106 and a flexible elongate member disposed in a slot of torque device 104 may lock torque device 104 to the flexible elongate member.

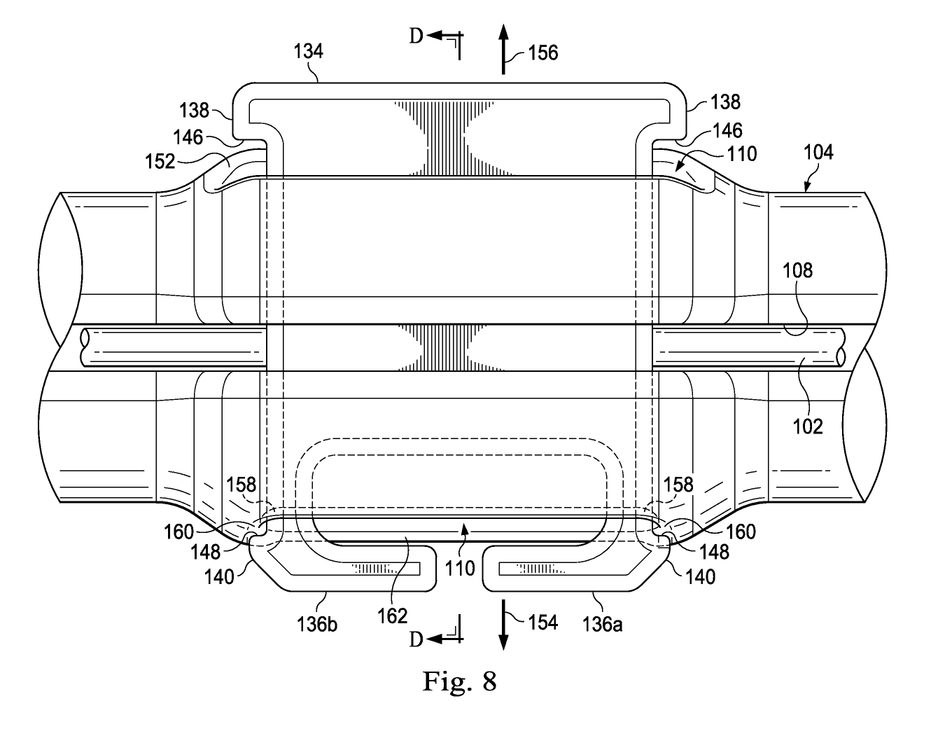

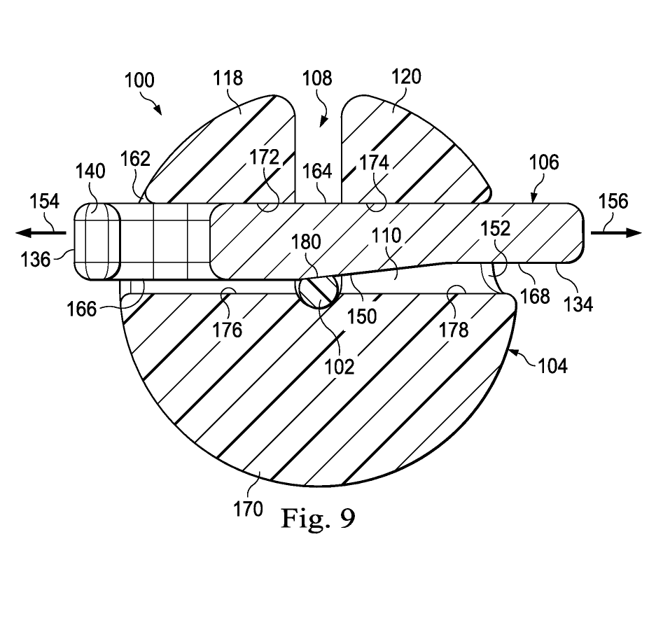

In the top view of FIG. 6, wedge 106 is shown to be generally rectangular. Wedge 106 includes a body 132, a right end 134, and a left end 136. In the discussion herein, left end 136 may be used to collectively refer to first or proximal section 136a, and second or distal section 136b. “Right” in right end 134 and “left” in left end 136 refer to relative directions when wedge 106 is received in bore 110. As shown in, e.g., FIGS. 8, 9, when wedge 106 locks torque device 104 to flexible elongate member 102, right end 134 of the wedge 106 is proximate to right opening 152 of torque device 104 and left end 136 is proximate to left opening 162. Wedge 106 may be referred to as part of a closing or fastening mechanism.

Referring again to FIG. 6, right end 134 includes protrusions 138 on both proximal and distal ends thereof. Each protrusion 138 includes an exterior contact surface 146. As described in the discussion of FIG. 8, contact surface 146 may contact interior contact surface 158 of torque device 104 during lateral translation of wedge 106 to prevent wedge 106 from being separated from torque device 104. Left end 136 may be divided into a first or distal section 136a and a second or proximal section 136b. First section 136a and second section 136b are separated by space 144. In some embodiments, left end 136 may be a unitary piece (and not divided into first section 136a and second section 136b, or separated by space 144). Similar to the right end 134, first section 136a and second section 136b of the left end include protrusions 140. Each protrusion 140 includes a contact surface 148. As described in the discussion FIG. 8, contact surfaces 148 may contact an exterior contact surface 160 during lateral translation of wedge 106 to prevent wedge 106 from being separated from torque device 104.

Wedge 106 includes opening 142 between body 132 and left end 136. Opening 142 and/or space 144 may be features that allow an operator to more easily grasp wedge 106. For example, an operator may use a thumb and pointer finger to grasp left end 136. Opening 142 and/or space 144 may provide textural variation for wedge 106 that eases an operator's ability to grasp, push, and/or pull wedge 106 to cause lateral translation of wedge 106. In other embodiments, other structural and/or textural features may be provided on wedge 106 in addition to or in lieu of opening 142 and/or space 144.

Referring now to FIG. 7, a diagrammatic back view of a wedge is shown, according to one embodiment of the present disclosure. Wedge 106 is shown to include body 132, left end 136 (e.g., first section 136a of FIG. 6), and right end 134. Protrusion 140 extends from left end 136, and protrusion 138 extends from right end 134. A top surface 164 of wedge 106 is flat (e.g., zero slope between left end 136 and right end 134). In some embodiments, left end 136 has a greater height than right end 134. To account for the differing heights of left end 136 and right end 134, a bottom surface of wedge 106 includes one or more sloped or tapered sections. For example, the bottom surface of wedge 106 may include sloped section 150. Sloped section 150 is disposed between two flat sections 166, 168 of the bottom surface. Flat section 166 is proximate to left end 136, and flat section 168 is proximate to right end 134. In some embodiments, the entire bottom surface of wedge 106 may be sloped or tapered. Sloped section 150 may contact a flexible elongate member to couple and/or lock torque device 104 to flexible elongate member 102. In the back view of wedge 106 shown in FIG. 7, sloped section 150 has a positive slope. In various embodiments, right end 134 may have a greater height than left end 136. In such embodiments, sloped section 150 may have a negative slope in the back view shown in FIG. 7.

Referring now to FIG. 8, a diagrammatic top view of an intravascular system is shown, according one embodiment of the present disclosure. Flexible elongate member 102 and torque device 104 are shown to be coupled together via wedge 106 in intravascular system 100. Flexible elongate member 102 is received in slot 108 of torque device 104. Wedge 106 is received in bore 110 while flexible elongate member 102 is in slot 108 such that at least a portion of the bottom surface of wedge 106 contacts a portion of flexible elongate member 102. As seen in FIG. 8, greater the length (along the longitudinal axis of torque device 104) of wedge 106, the more surface area of contact exists between flexible elongate member 102 and the wedge.

Wedge 106 may translate laterally in directions 154, 156. That is, wedge 106 may translate transverse to slot 108 within bore 110 such that slot 108 is selectively open so that torque device 104 may be loaded onto flexible elongate member 102 and selectively closed so that torque device 104 is locked to flexible elongate member 102. An operator may cause wedge 106 to be laterally translated by pushing and/or pulling wedge 106 in directions 154, 156. For example, an operator may use a thumb and pointer finger to grasp left end 136, pull wedge 106 in direction 154, and/or push wedge 106 in direction 156.

In some embodiments, wedge 106 may be inserted into bore 110 during manufacture of torque device 104. In some embodiments, wedge may be coupled to or separated from bore 110 by an operator of torque device 104 during use thereof. In some embodiments, after wedge 106 is inserted into bore 110, the wedge 106 is advantageously prevented from being separated from torque device 104 while simultaneously allowed translate within bore 110.

Prior to torque device 104 being loaded onto flexible elongate member 102, wedge 106 may be laterally translated in direction 154 such that right end 134 is brought adjacent to left opening 162 of torque device 104. (See, for example, FIG. 10.) The length (e.g., along the longitudinal axis of torque device 104) of right opening 152 is larger than the length of right end 134 of wedge 106. Accordingly, right end 134 is able to clear the right opening 152 when wedge 106 is translated in direction 154, without contact between wedge 106 and torque device 104. When right end 134 of wedge 106 is adjacent to left opening 162 of torque device 104, slot 108 of the torque device is open such that at least a portion of flexible elongate member 102 may be received in slot 108. That is, torque device 104 may be side loaded onto flexible elongate member 102 because an entire lateral side of torque device 104 is open (e.g., slot 108). When slot 108 is open, left end 136, opening 142, and body 132 of wedge 106 may be outside of torque device 104. This is shown, for example, in FIG. 10, in which portions of wedges 206, 216 are shown to be outside of torque devices 204, 214, respectively. Note that the slots of torque devices 204, 214 are unimpeded such that flexible elongate members 202 and 212 are received respectively therein.

Referring again to FIG. 8, wedge 106 may be structured such that the wedge 106 advantageously remains coupled to torque device 104 while an operator is handling torque device 104 and/or wedge 106. The length of left opening 162 may be less than the length of the right end 134 of wedge 106 (including protrusions 138). As such, right end 134 is not able to clear left opening 162 when wedge 106 is translated in direction 154. Thus, there is contact between wedge 106 and torque device 104 when wedge 106 is attempted to be translated in direction 154 beyond left opening 162. In that regard, contact surfaces 146 of protrusions 138 may contact interior contact surface 158 of left opening 162. Contact between wedge 106 and torque device 104 advantageously prevents wedge 106 from falling out of bore 110 and being separated from torque device 104. This aspect of the present disclosure may be described a retaining feature of torque device 104 because the feature retains wedge 106 within a volume of torque device 104 even when flexible elongate member 102 is not coupled to torque device 104.

Once torque device 104 is loaded onto a portion of flexible elongate member 102 (e.g., such that flexible elongate member 102 is received in slot 108), wedge 106 may be translated in direction 156, over flexible elongate member 102, to couple and/or lock torque device 104 and flexible elongate member 102. When wedge 106 is translated in direction 156, right end 134 passes over the top of slot 108 and flexible elongate member 102. Right end 134 clears right opening 152, as described above. Wedge 106 advantageously remains coupled to torque device 104 because left end 136 is prevented from translating past left opening 162 as wedge 106 is translated in direction 156. The length of left opening 162 may be less than the length of the left end 136 of wedge 106 (including protrusions 140). As such, left end 136 is not able to clear left opening 162 when wedge 106 is translated in direction 156. Thus, there is contact between wedge 106 and torque device 104 when wedge 106 is attempted to be translated in direction 156 beyond left opening 162. In that regard, contact surfaces 148 of protrusions 140 may contact exterior contact surface 158 of left opening 162. Contact between wedge 106 and torque device 104 advantageously prevents wedge 106 from falling out of bore 110 and being separated from torque device 104.

Referring now to FIG. 9, a diagrammatic cross-sectional back view of an intravascular system is shown, according to one embodiment of the present disclosure. FIG. 9 is a cross-sectional view of intravascular system 100 along section line D-D of FIG. 8. Flexible elongate member 102 is shown to be received in slot 108 of torque device 104. In one embodiment of the present disclosure, wedge 106 may be translated within bore 110 in direction 154 in order to open slot 108 such that torque device 104 maybe loaded onto a portion of flexible elongate member 102. Once flexible elongate member 102 is received in slot 108, wedge 106 may be translated within bore 110 in direction 156 to couple and/or lock flexible elongate member 102 and torque device 104.

As wedge 106 is translated in direction 156, contact occurs between sloped portion 150 of the bottom surface of wedge 106 and flexible elongate member 102. The farther wedge 106 is translated in direction 156, the more contact occurs between sloped portion 150 and flexible elongate member 102 and the more force is applied to flexible elongate member 102. As more contact and force are applied to guide 102, flexible elongate member 102 because further engaged with slot 108 (e.g., forced into contact with a surface of slot 108). As a result of the contact and force acting on flexible elongate member 102, flexible elongate member 102 is coupled and/or locked to torque device 104. Accordingly, e.g., rotation of torque device 104 about the torque device's longitudinal axis causes rotation of flexible elongate member 102 about the flexible elongate member's longitudinal axis. Because sloped portion 150 is in fact sloped, there is advantageously greater surface area of contact between wedge 106 and flexible elongate member 102 compared to contact between a zero slope surface and flexible elongate member 102. To uncouple and/or unlock flexible elongate member 102 and torque device 104, wedge 106 may be translated in direction 154 such that contact between flexible elongate member 102 and wedge 106 is lessened or eliminated.

The coupling and/or locking of flexible elongate member 102 and torque device 104 is facilitated in part by contact between a top surface 164 of wedge 106 and surfaces 172 and 174 of torque device as wedge 106 translates in direction 156. Surface 172 is a top surface of bore 110 in the left portion 118. Surface 174 is a top surface of bore 110 in the right portion 120. Contact may also occur between flat portion 166 of the bottom surface of wedge 106 and surface 176 of torque device 176. Surface 176 is a bottom surface of bore 110 in the left portion 118. Because the height of left end 136 is greater than right end 134 and because of the at least partially sloped bottom surface of wedge 106, contact is not made between flat surface 168 of wedge 106 and surface 178 of torque device 104. Surface 178 is a bottom surface of bore 110 in the right portion 120. When flexible elongate member 102 is coupled and/or locked to torque device 104, left end 136 may extend beyond left opening 162 and right end 134 may extend beyond right opening 152.

The orientation of features shown and described is exemplary only. In other embodiments, the features may be disposed in a different orientation. For example, in FIG. 6, right end 134 may be split into a first portion and a second portion (as left end 136 is shown to be). Similarly, e.g., in FIG. 9, sloped portion 150 may have negative slope, and wedge 106 may be translated in direction 154 to couple and/or lock flexible elongate member 102 and torque device 104 together.

Referring now to FIG. 10, diagrammatic perspective views of intravascular systems are shown, according to embodiments of the present disclosure. Intravascular system 200 includes flexible elongate member 202, torque device 204, and wedge 206. Intravascular system 210 includes flexible elongate member 212, torque device 214, and wedge 216. In FIG. 10, intravascular systems 200, 210 are shown after torque devices 204, 214 has been side-loaded onto respective portions of flexible elongate members 202, 212. Flexible elongate member 202 is shown to have a larger radius than flexible elongate member 212. A slot for receiving the flexible elongate member in the torque device may be sized and shaped to receive guide wires of differing sizes. Accordingly, the slot for receiving the flexible elongate member in torque device 214 is narrower than the slot in torque device 204. FIG. 10 shows wedges 206, 216 in the open position. As described above, wedges 206, 216 translate within respective bores of torque devices 204, 214. When the wedges are in the open position, they are recessed into respective retaining features (as described in the discussion of FIG. 8) and the entire slot is open for wire insertion. That is, slots for receiving flexible elongate members 202, 212 are unimpeded as wedges 206, 216 are translated out of the way of the slots. Portions of wedge 206, 216 are outside of torque devices 204, 214 when the slots for receiving guide wires 202, 212 are made clear (e.g., portions of wedges 206, 216 extend beyond the volumes of torque devices 204, 214). As described in the discussion of FIG. 8, one or more retention features of wedges 206, 216 enable wedges to remain coupled to torque devices 204, 214, respectively, even when the portions of wedge 206, 214 are hanging outside torque devices 204, 214.

Referring now to FIG. 11, diagrammatic perspective views of intravascular systems are shown, according to embodiments of the present disclosure. FIG. 11 shows wedges 206, 215 in a closed position. To couple and/lock guide wires 202, 212 and torque devices 204, 214, respectively, wedges 206, 216 are translated at least partially transverse to the length of the guide wires 202, 212. When wedges 206, 216 are thus translated, there is contact between bottom surfaces of wedges 206, 216 and guide wires 202, 212, respectively. The contact results in force being applied to guide wires 202, 212, which causes guide wires 202, 212 to be further engaged with longitudinal slots of torque devices 204, 214. When torque devices 204, 214 are being coupled to flexible elongate member 202, 212, FIG. 11 is later in time than FIG. 10. When torque devices 204, 214 are being uncoupled from flexible elongate member 202, 212, FIG. 10 is later in time than FIG. 11.

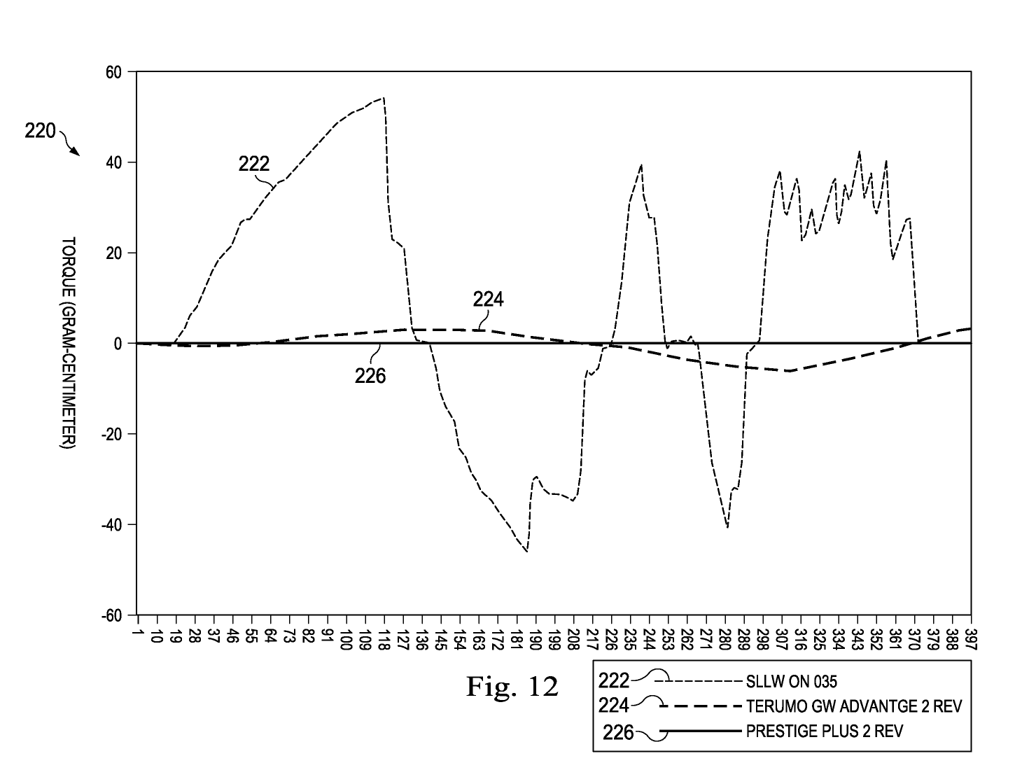

Referring now to FIG. 12, a chart describing torqueability of an intravascular system is shown, according to one embodiment of the present disclosure. In this embodiment, torqueability is measured as the maximum torque achieved on a flexible elongate member before the flexible elongate member slipped. “Slipped” may refer to the rotation of flexible elongate member about its longitudinal axis or the cessation of such rotation, independent of the rotation of torque device about the torque device's longitudinal axis. Recall that when flexible elongate member and torque device are locked, rotation of flexible elongate member should follow rotation of the torque device. The higher the maximum torque before slippage, the stronger the coupling between the flexible elongate member and torque device. With a high torqueability, an operator can rotate torque device with relatively high confidence that the flexible elongate member will correspondingly rotate.

The results shown in the chart 200 are from testing performed using a 0.035″ polyimide coated composite wire (curve 222) and two other wires (curves 224 and 226). The y-axis of chart 220 is torque in units of gram-centimeter. The testing was performed with saline on the wire/connector to simulate actual use conditions. The wires represented by curves 224 and 226 simulate conditions when an intravascular device becomes lodged or stuck within an anatomy of a patient. During testing, such conditions are simulated by locking a distal tip of the wires represented by curves 224 and 226 such that the distal tip cannot be rotated. The proximal ends of the wires are then rotated, and the torque generated in the wires is measured. The wires represented by curves 224 and 226 were rotated two full revolutions in one direction, then rotated back to zero, and then rotated two full revolutions in the other direction. Revolutions in one direction are indicated by the positive values of, e.g., curve 224, and revolutions in the other direction are indicated by the negatives values of, e.g., curve 224. The torque that is generated in the wires represented by curves 224 and 226 under these conditions is more torque than would usually be generated during normal operation (because the distal end of the wires are not usually prevented from rotating during normal operations). These results associated with the test for each wire were overlaid with the results from the test of the 0.035″ composite wire described below to show the comparison of how much torque can be generated with the torque device described herein as compared to how much torque is generated by a wire in which the distal tip is locked while rotating the proximal end.

The 0.035″ composite wire was rotated, using the torque device described herein, until the wire slipped. Curve 222 shows that the initial torque prior to slippage on the wedge prototype was approximately 54 g-cm. This torque is much greater than the torque measured under the simulated conditions represented by curve 224 and 226. As described above, the torque shown in curves 224 and 226 is greater than torque that would occur in wires under normal operating conditions. Because curve 222 shows that an even greater torque can be achieved than under the conditions represented curves 224 and 226, the torque device described herein is efficacious in providing high torqueability to an intravascular device. A high torqueability advantageously provides a large safety margin for the response of the flexible elongate member coupled to the torque device described herein when the torque device is rotated. Selection of wedge/body materials, as well as variation in wedge design may allow for even higher torque values to be generated.

In view of all of the above and the figures, one of ordinary skill in the art will readily recognize that the present disclosure introduces a torque device. The torque device includes a body having a proximal portion, a distal portion, and a longitudinal axis. The body includes a slot extending along a length of the body parallel to the longitudinal axis. The slot extends from an exterior surface of the body to an interior surface of the body. The slot is sized and shaped to receive a flexible elongate member. The body includes an opening extending through the body perpendicular to the longitudinal axis and in communication with the slot. The torque device includes a closing mechanism movably coupled to the body. The closing mechanism is movable within the opening of the body between an open position that allows the flexible elongate member to be inserted into the slot and a locked position that fixedly secures the flexible elongate member to the torque device.

In some embodiments, the closing mechanism is translatable within the opening of the body in a direction perpendicular to the longitudinal axis of the body between the open and locked positions. In some embodiments, the closing mechanism includes a wedge component. In some embodiments, the wedge component includes a first surface and an opposing second surface, the second surface extending at an oblique angle with respect to the first surface. In some embodiments, the second surface is configured to urge the flexible elongate member against the interior surface of the body as the closing mechanism is moved between the open position and the locked position. In some embodiments, engagement of the flexible elongate member with the second surface of the wedge component and the interior surface of the body fixedly secures the flexible elongate member to the torque device when the closing mechanism is in the locked position. In some embodiments, the interior surface of the body is positioned such that the flexible elongate member is coaxially disposed with the body when fixedly secured to the torque device by the closing mechanism. In some embodiments, the slot is configured to receive the flexible elongate member in a direction perpendicular to the longitudinal axis of the body. In some embodiments, the closing mechanism is further movable within the opening of the body to an intermediate position between the open and locked positions, wherein in the intermediate position a flexible elongate member positioned within the slot is movable with respect to the torque device but cannot be removed from the slot in a direction perpendicular to the longitudinal axis of the body. In some embodiments, the flexible elongate member positioned within the slot is translatable with respect to the torque device along the longitudinal axis of the body when the closing mechanism is in the intermediate position. In some embodiments, the closing mechanism includes an engagement feature to prevent separation of the closing mechanism from the body. In some embodiments, the engagement feature is at least one projection.

The present disclosure also introduces a system. The system includes an intravascular device sized and shaped for insertion within a vessel of a patient. The system includes a torque device configured to selectively, fixedly engage a proximal section of the intravascular device. The torque device includes a body having a proximal portion, a distal portion, and a longitudinal axis. The body includes a slot extending along a length of the body parallel to the longitudinal axis. The slot extends from an exterior surface of the body to an interior surface of the body. The slot is sized and shaped to receive at least the proximal section of the intravascular device. The body includes an opening extending through the body perpendicular to the longitudinal axis and in communication with the slot. The torque device includes a closing mechanism movably coupled to the body. The closing mechanism is movable within the opening of the body between an open position that allows the intravascular device to be inserted into the slot and a locked position that fixedly secures the intravascular device to the torque device.

In some embodiments, the intravascular device is a guide wire. In some embodiments, the intravascular device is a catheter. In some embodiments, the intravascular device is a pressure-sensing device. In some embodiments, the intravascular device is an imaging device. In some embodiments, intravascular device is a flow-sensing device.

The present disclosure also introduces a method. The method includes inserting an intravascular device into a slot of a body portion of a torque device in a direction perpendicular to a longitudinal axis of the intravascular device. The method includes moving a closing mechanism of the torque device from an open position that allows the intravascular device to be inserted into the slot to a locked position that fixedly secures the intravascular device to the torque device. Moving the closing mechanism from the open position to the closed position includes translating the closing mechanism in a direction perpendicular to the longitudinal axis of the intravascular device along an opening in the body portion of the torque device that is in communication with the slot.

In some embodiments, the closing mechanism includes a wedge component having a first surface and an opposing second surface extending at an oblique angle with respect to the first surface such that moving the closing mechanism from the open position to the locked position causes the second surface to urge the intravascular device against an interior surface of the body portion bounding the slot such that engagement of the intravascular device with the second surface of the wedge component and the interior surface of the body portion fixedly secures the intravascular device to the torque device when the closing mechanism is in the locked position.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure.

Accordingly, it is appropriate that the appended claims be construed broadly and in a manner consistent with the present disclosure.

What is claimed is:

1. A torque device, comprising:
a body having a proximal portion, a distal portion, and a longitudinal axis, the body including:
a slot extending along a length of the body parallel to the longitudinal axis, the slot extending from an exterior surface of the body to an interior surface of the body, wherein the slot is sized and shaped to receive a flexible elongate member; and
an opening extending through the body perpendicular to the longitudinal axis and in communication with the slot; and
a closing mechanism movably coupled to the body, wherein the closing mechanism is movable within the opening of the body between an open position that allows the flexible elongate member to be inserted into the slot and a locked position that fixedly secures the flexible elongate member to the torque device.

2. The torque device of claim 1, wherein the closing mechanism is translatable within the opening of the body in a direction perpendicular to the longitudinal axis of the body between the open and locked positions.

3. The torque device of claim 2, wherein the closing mechanism includes a wedge component.

4. The torque device of claim 3, wherein the wedge component includes a first surface and an opposing second surface, the second surface extending at an oblique angle with respect to the first surface.

5. The torque device of claim 4, wherein the second surface is configured to urge the flexible elongate member against the interior surface of the body as the closing mechanism is moved between the open position and the locked position.

6. The torque device of claim 5, wherein engagement of the flexible elongate member with the second surface of the wedge component and the interior surface of the body fixedly secures the flexible elongate member to the torque device when the closing mechanism is in the locked position.

7. The torque device of claim 6, wherein the interior surface of the body is positioned such that the flexible elongate member is coaxially disposed with the body when fixedly secured to the torque device by the closing mechanism.

8. The torque device of claim 1, wherein the slot is configured to receive the flexible elongate member in a direction perpendicular to the longitudinal axis of the body.

9. The torque device of claim 1, wherein the closing mechanism is further movable within the opening of the body to an intermediate position between the open and locked positions, wherein in the intermediate position a flexible elongate member positioned within the slot is movable with respect to the torque device but cannot be removed from the slot in a direction perpendicular to the longitudinal axis of the body.

10. The torque device of claim 9, wherein the flexible elongate member positioned within the slot is translatable with respect to the torque device along the longitudinal axis of the body when the closing mechanism is in the intermediate position.

11. The torque device of claim 1, wherein the closing mechanism includes an engagement feature to prevent separation of the closing mechanism from the body.

12. The torque device of claim 11, wherein the engagement feature is at least one projection.

13. A system, comprising:
an intravascular device sized and shaped for insertion within a vessel of a patient; and
a torque device configured to selectively, fixedly engage a proximal section of the intravascular device, the torque device including:
a body having a proximal portion, a distal portion, and a longitudinal axis, the body including:
a slot extending along a length of the body parallel to the longitudinal axis, the slot extending from an exterior surface of the body to an interior surface of the body, wherein the slot is sized and shaped to receive at least the proximal section of the intravascular device; and
an opening extending through the body perpendicular to the longitudinal axis and in communication with the slot; and
a closing mechanism movably coupled to the body, wherein the closing mechanism is movable within the opening of the body between an open position that allows the intravascular device to be inserted into the slot and a locked position that fixedly secures the intravascular device to the torque device.

14. The system of claim 13, wherein the intravascular device is a guide wire.

15. The system of claim 13, wherein the intravascular device is a catheter.

16. The system of claim 13, wherein the intravascular device is a pressure-sensing device.

17. The system of claim 13, wherein the intravascular device is an imaging device.

18. The system of claim 13, wherein the intravascular device is a flow-sensing device.

19. A method, comprising:
inserting an intravascular device into a slot of a body portion of a torque device in a direction perpendicular to a longitudinal axis of the intravascular device;
moving a closing mechanism of the torque device from an open position that allows the intravascular device to be inserted into the slot to a locked position that fixedly secures the intravascular device to the torque device, wherein moving the closing mechanism from the open position to the closed position includes translating the closing mechanism in a direction perpendicular to the longitudinal axis of the intravascular device along an opening in the body portion of the torque device that is in communication with the slot.

20. The method of claim 19, wherein the closing mechanism includes a wedge component having a first surface and an opposing second surface extending at an oblique angle with respect to the first surface such that moving the closing mechanism from the open position to the locked position causes the second surface to urge the intravascular device against an interior surface of the body portion bounding the slot such that engagement of the intravascular device with the second surface of the wedge component and the interior surface of the body portion fixedly secures the intravascular device to the torque device when the closing mechanism is in the locked position.
